(19) United States
(12) Patent Application Publication
(10) Pub. No.: US 2025/0339177 A1
(43) Pub. Date: Nov. 6, 2025

(54) Medical Devices and Kits Useful for Performing Treatment under Magnetic Resonance Imaging and Related Methods

(71) Applicant: Cook Medical Technologies LLC, Bloomington, IN (US)

(72) Inventors: Neal Fearnot, West Lafayette, IN (US); Sean D. Chambers, Bloomington, IN (US); Gary L. Neff, Bloomington, IN (US); Ram H. Paul, Bloomington, IN (US); Eric Brandner, West Lafayette, IN (US)

(73) Assignee: Cook Medical Technologies LLC, Bloomington, IN (US)

(21) Appl. No.: 19/273,381

(22) Filed: Jul. 18, 2025

Related U.S. Application Data
(63) Continuation of application No. 17/573,087, filed on Jan. 11, 2022.
(60) Provisional application No. 63/135,801, filed on Jan. 11, 2021.

(51) Int. Cl. A61B 17/34; A61B 10/02; A61B 17/00; A61B 90/00

(52) U.S. Cl. CPC A61B 17/3421; A61B 10/0241; A61B 17/00234; A61B 90/36; A61B 2017/00274; A61B 2017/3456; A61B 2017/3484; A61B 2090/374

(57) ABSTRACT

Example medical devices for performing treatment under magnetic resonance imaging and related methods are described. An example medical device includes a cannula and a plug. The cannula has a proximal end, a distal end, a first portion, a second portion, and a main body that defines a lumen and a passageway. The first portion extends from the distal end toward the proximal end. The second portion extends from the first portion toward the proximal end. The first portion is formed of first material. The second portion is formed of a second material that is different than the first material. The plug is disposed within the passageway and has a main body that defines a passageway in fluid communication with the lumen defined by the cannula. The plug is formed of a third material that is different than the second material.

### RELATED APPLICATIONS

This application is a continuation of U.S. patent application Ser. No. 17/573,087, filed Jan. 11, 2022, which claims the benefit of U.S. Provisional Application No. 63/135,801, filed Jan. 11, 2021. The entire contents of these related applications are hereby incorporated by reference into this disclosure.

### FIELD

The disclosure relates generally to the field of medical devices. More particularly, the disclosure relates to medical devices useful in performing treatment under magnetic resonance imaging (MRI), kits useful in performing treatment under MRI, and methods of performing interventional medical treatment under MRI.

### BACKGROUND

The field of interventional MRI is gaining wider acceptance and seeing an increase in the number of procedures that can be performed. Interventional procedures conducted under MRI have several benefits over X-Ray-guided interventions. For example, the patient is not exposed to ionizing radiation. Also, MRI provides the ability to characterize tissue and functional flow during an interventional procedure.

The development of interventional procedures conducted under MRI has been limited as a result of the tools needed to perform these procedures being unavailable. Therefore, patients are required to make multiple visits to treatment facilities to visualize, diagnose, and treat various conditions. In addition, multiple imaging modalities are often needed, which impacts the accuracy of utilizing a magnetic resonance image in directing intervention. For example, when addressing prostate cancer, visualization, biopsy, and treatment are currently completed over the course of three patient visits. At a first visit, a scan is completed using a magnetic resonance scanner to produce an image showing the prostate and any abnormalities. The patient then leaves the facility and awaits a review of the image. If abnormalities exist, a second patient visit will occur such that a biopsy sample of the abnormal tissue can be completed. Currently, software is used to fuse the magnetic resonance image with the procedural ultrasound to provide guidance in conducting the biopsy. This fusion decreases the value of the diagnostic magnetic resonance image. The patient then leaves the facility again and awaits a review of the biopsy sample to determine whether further treatment is required (e.g., if the review results in a positive prostate cancer diagnosis). If further treatment is required, the patient will visit the facility a third time such that treatment can be performed. Completion of these three patient visits can take months, prevents the patient from receiving rapid treatment, and increases the overall costs associated with treatment. Furthermore, software used to fuse magnetic resonance images with other images (e.g., those obtained via ultrasound) have drawbacks, such as potential image overlay issues and the potential for compression shifting of tissues (e.g., prostate).

A need exists, therefore, for new and improved medical devices useful in performing treatment under MRI, kits useful in performing treatment under MRI, and methods of performing interventional medical treatment under MRI.

### SUMMARY OF SELECTED EXAMPLE EMBODIMENTS

Various example medical devices useful in performing treatment under MRI, kits useful in performing treatment under MRI, and methods of performing interventional medical treatment under MRI are described herein.

An example medical device useful in performing treatment under MRI includes a cannula and a plug. The cannula has a proximal end, a distal end, a first portion, a second portion, and a main body that defines a lumen and a passageway. The first portion extends from the distal end toward the proximal end. The second portion extends from the first portion toward the proximal end. The first portion is formed of a first material. The second portion is formed of a second material that is different than the first material. The lumen extends from the proximal end to the distal end. The passageway is defined on the second portion and extends through the main body and is in fluid communication with the lumen. The plug is disposed within the passageway. The plug has a main body that defines a passageway in fluid communication with the lumen defined by the cannula. The plug is formed of a third material that is different than the second material.

An example method of performing an interventional medical treatment under MRI comprises positioning a patient within a magnetic resonance scanner; scanning a first portion of the patient using the magnetic resonance scanner; obtaining a magnetic resonance image of the first portion of the patient; identifying a tissue that has predefined characteristics using the magnetic resonance image; while the patient remains positioned within the magnetic resonance scanner used to scan the first portion of the patient, advancing a medical device into a bodily passage and to the tissue while scanning a second portion of the patient that includes the medical device using the magnetic resonance scanner; obtaining a magnetic resonance image of the second portion of the patient that includes the medical device; confirming the position of the medical device within the bodily passage; advancing a biopsy device through the medical device and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the medical device; and determining whether the tissue sample meets a predefined criterion.

Another example method of performing an interventional medical treatment comprises positioning a patient within a magnetic resonance scanner; scanning a first portion of the patient using the magnetic resonance scanner; obtaining a magnetic resonance image of the first portion of the patient; identifying a tissue that has predefined characteristics using the magnetic resonance image; while the patient remains positioned within the magnetic resonance scanner used to scan the first portion of the patient, advancing a medical device into a bodily passage and to the tissue while scanning a second portion of the patient that includes the medical device using the magnetic resonance scanner; obtaining a magnetic resonance image of the second portion of the patient that includes the medical device; confirming the position of the medical device within the bodily passage; advancing a biopsy device through the medical device and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the medical device; determining whether the tissue sample meets a predefined criterion; if the tissue sample does not meet the predefined criterion, additional steps comprise withdrawing the medical device from the bodily passage and removing the patient from the magnetic resonance scanner; if the tissue sample meets the predefined criterion, advancing an anchor member through the medical device through which the biopsy device was advanced and to the tissue while the patient remains positioned within the magnetic resonance scanner; securing the anchor member to retain the position of the medical device relative to the tissue; advancing a first inner sheath over the medical device and toward the tissue to dilate the bodily passage; advancing a second inner sheath over the first inner sheath and toward the tissue to dilate the bodily passage; advancing an outer sheath over the second inner sheath and toward the tissue to dilate the bodily passage; removing the anchor member from the tissue; withdrawing the anchor member from the bodily passage; withdrawing the medical device from the bodily passage; withdrawing the first inner sheath from the bodily passage; withdrawing the second inner sheath from the bodily passage; advancing a treatment device through the outer sheath and to the tissue; manipulating the tissue using the treatment device; withdrawing the treatment device from the outer sheath; and withdrawing the outer sheath from the bodily passage.

An example method of performing treatment on a prostate under MRI comprises positioning a patient within a magnetic resonance scanner; scanning a prostate and surrounding tissue of the patient using the magnetic resonance scanner; obtaining a magnetic resonance image of the prostate and surrounding tissue of the patient; identifying a tissue within the magnetic resonance image that has predefined characteristics; while the patient remains positioned within the magnetic resonance scanner used to scan the prostate and surrounding tissue, advancing a medical device into a bodily passage and to the tissue while scanning a first portion of the patient that includes the medical device using the magnetic resonance scanner; obtaining a magnetic resonance image of the second portion of the patient that includes the medical device; confirming the position of the medical device within the bodily passage; advancing a biopsy device through the medical device and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the medical device; determining whether the tissue sample meets a predefined criterion; if the tissue sample meets the predefined criterion, advancing an anchor member through the medical device through which the biopsy device was advanced and to the tissue while the patient remains positioned within the magnetic resonance scanner; securing the anchor member to the tissue to retain the position of the medical device relative to the tissue; advancing a first inner sheath over the medical device and toward the tissue to dilate the bodily passage; advancing a second inner sheath over the first inner sheath and toward the tissue to dilate the bodily passage; advancing an outer sheath over the second inner sheath and toward the tissue to dilate the bodily passage; removing the anchor member from the tissue; withdrawing the anchor member from the bodily passage; withdrawing the medical device from the bodily passage; withdrawing the first inner sheath from the bodily passage; withdrawing the second inner sheath from the bodily passage; advancing a treatment device through the outer sheath and to the tissue; manipulating the tissue using the treatment device; withdrawing the treatment device from the outer sheath; withdrawing the outer sheath from the bodily passage.

Another example method of performing an interventional medical treatment comprises positioning a patient within a magnetic resonance scanner; scanning a first portion of the patient using the magnetic resonance scanner; obtaining a magnetic resonance image of the first portion of the patient; identifying a tissue that has predefined characteristics using the magnetic resonance image; while the patient remains positioned within the magnetic resonance scanner used to scan a portion of the patient, advancing a medical device into a bodily passage and to the tissue while scanning a second portion of the patient that includes the medical device using the magnetic resonance scanner; obtaining a magnetic resonance image of the second portion of the patient that includes the medical device; confirming the position of the medical device within the bodily passage; advancing an anchor member through the medical device and to the tissue while the patient remains positioned within the magnetic resonance scanner; securing the anchor member to the tissue to retain the position of the anchor member relative to the tissue; withdrawing the medical device from the bodily passage; advancing a first inner sheath over the anchor member and toward the tissue to dilate the bodily passage; advancing a second inner sheath over the first inner sheath and toward the tissue to dilate the bodily passage; advancing an outer sheath over the second inner sheath and toward the tissue to dilate the bodily passage; withdrawing the first inner sheath from the bodily passage; withdrawing the second inner sheath from the bodily passage; removing the anchor member from the tissue; withdrawing the anchor member from the bodily passage; advancing a biopsy device through the outer sheath and to the tissue while scanning a third portion of the patient that includes the biopsy device using the magnetic resonance scanner; obtaining a magnetic resonance image of the third portion of the patient that includes the biopsy device; confirming the position of the biopsy device; collecting a tissue sample from the tissue using the biopsy device while scanning a fourth portion of the patient that includes the biopsy device and the tissue using the magnetic resonance scanner; obtaining a magnetic resonance image of the fourth portion of the patient that includes the biopsy device; confirming the tissue sample has been collected; withdrawing the biopsy device and the tissue sample through the outer sheath; determining whether the tissue sample meets a predefined criterion; if the tissue sample does not meet the predefined criterion, withdrawing the outer sheath from the bodily passage; removing the patient from the magnetic resonance scanner; if the tissue sample meets the predefined criterion, advancing a treatment device through the outer sheath and to the tissue; manipulating the tissue using the treatment device; withdrawing the treatment device from the outer sheath; withdrawing the outer sheath from the bodily passage; removing the patient from the magnetic resonance scanner.

An example kit useful in performing treatment under magnetic resonance includes a cannula, a first inner sheath, a second inner sheath, an outer sheath, an anchor member, and a treatment device.

Additional understanding of these example medical devices, kits, and methods can be obtained by review of the detailed description, below, and the appended drawings.

### BRIEF DESCRIPTION OF THE DRAWINGS

FIG. 1 is a partial elevation view of a first example medical device.

FIG. 2 is a partial elevation view of the first medical device illustrated in FIG. 1 disposed through a first inner sheath, a second inner sheath, and an outer sheath.

FIG. 3 is a partial elevation view of the outer sheath illustrated in FIG. 2.

FIG. 4 is a partial perspective view of a second example medical device.

FIG. 5 is a partial perspective view of a third example medical device.

FIG. 6 is a partial perspective exploded view of a fourth example medical device.

FIG. 7 is an elevation view of a fifth example medical device.

FIG. 8 is a partial elevation view of a sixth example medical device.

FIG. 9 is a partial sectional view of a seventh example medical device. The anchor member is shown in a first position.

FIG. 10 is another partial sectional view of the medical device shown in FIG. 9. The anchor member is shown in a second position.

FIG. 11 is another partial sectional view of the medical device shown in FIG. 9. The anchor member is shown in a third position.

FIG. 12 is a partial top view of the cannula of the medical device shown in FIG. 9.

FIG. 13 is a partial elevation view of the cannula of the medical device shown in FIG. 9.

FIG. 14 is a partial top view of the anchor member of the medical device shown in FIG. 9.

FIG. 15 is a partial top view of an eighth example medical device. The anchor member is shown in a first position.

FIG. 16 is another partial top view of the medical device shown in FIG. 15. The anchor member is shown in a second position.

FIG. 17 is another partial top view of the medical device shown in FIG. 15. The anchor member is shown in a third position.

FIG. 18 is a partial elevation view of a ninth example medical device. The anchor member is shown in a first position.

FIG. 19 is another partial elevation view of the medical device shown in FIG. 18. The anchor member is shown in a second position.

FIG. 20 is another partial elevation view of the medical device shown in FIG. 18. The anchor member is shown in a third position.

FIG. 21 is a schematic illustration of an example method of performing treatment under MRI.

FIGS. 22A and 22B show another schematic illustration of an example method of performing treatment under MRI.

FIG. 23 is a partial perspective view of a tenth example medical device.

FIGS. 24A and 24B show another schematic illustration of an example method of performing treatment under MRI.

FIG. 25 illustrates an example kit that includes example medical devices, an example first inner sheath, an example second inner sheath, an example outer sheath, and an example treatment device.

FIGS. 26A and 26B show another schematic illustration of an example method of performing treatment under MRI.

### DETAILED DESCRIPTION OF SELECTED EXAMPLES

The following detailed description and the appended drawings describe and illustrate various example medical devices useful in performing treatment under MRI, kits useful in performing treatment under MRI, and methods of performing interventional medical treatment under MRI. The description and illustration of these examples are provided to enable one skilled in the art to make and use a medical device, a kit, and to practice a method of performing an interventional medical treatment under MRI. They are not intended to limit the scope of the invention, or the protection sought, in any manner. The invention is capable of being practiced or carried out in various ways and the examples described and illustrated herein are merely selected examples of the various ways of practicing or carrying out the invention and are not considered exhaustive.

As used herein, the term “attached” refers to one member being secured to another member such that the members do not completely separate from each other during use performed in accordance with the intended use of an item that includes the members in their attached form.

As used herein, the term “plug” refers to a member having a size and configuration suitable for disposition within a hole, cavity, passageway, or void in another member. The term does not require any particular size or configuration, and the size and configuration of a particular plug will depend on the size and configuration of the hole, cavity, passageway, or void into which the plug is intended to be disposed.

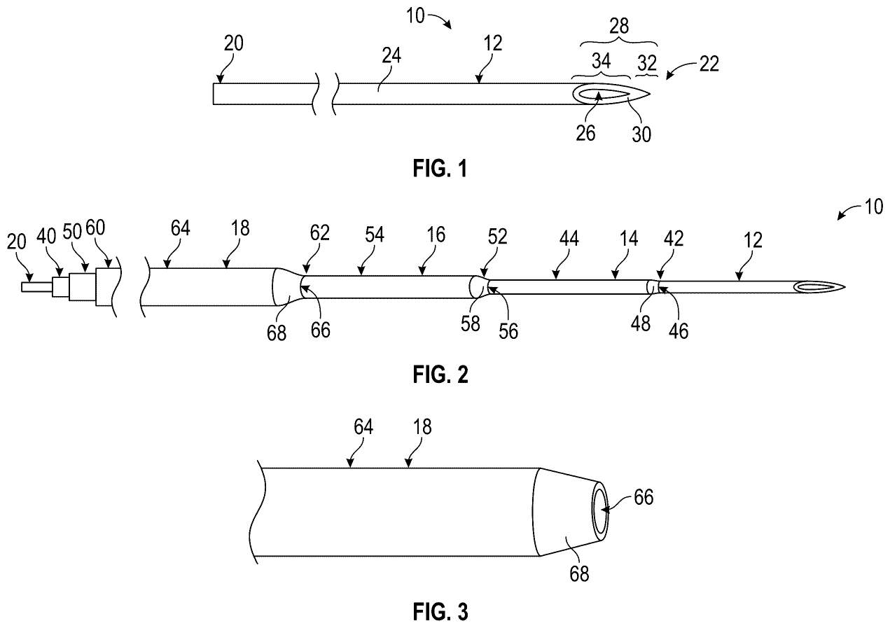

FIGS. 1, 2, and 3 illustrate a first example medical device 10. In this example, the medical device 10 is a cannula 12. As shown in FIG. 2, the cannula 12 can be used with a first inner sheath 14, a second inner sheath 16, and an outer sheath 18 to accomplish sequential dilation of a bodily passage to provide access to the bodily passage.

The cannula 12 has a proximal end 20, a distal end 22, and a main body 24 that defines a lumen 26 and a distal tapered tip 28 with a cutting edge 30. The lumen 26 extends from the proximal end 20 to the distal end 22 such that one or more devices can be passed into, and through, the cannula 12. In the illustrated embodiment, the main body 24 has a first portion 32 formed of a first material and a second portion 34 formed of a second material. The first portion 32 extends from the distal end 22 toward the proximal end 20 and the second portion 34 extends from the first portion 32 toward the proximal end 20. The first material and the second material can comprise any suitable MRI compatible material. For example, a first portion can comprise a magnetically susceptible material, such as a paramagnetic material or a ferromagnetic material, and the second portion can comprise a non-ferromagnetic material, such as an austentic nickel-chromium based alloy, such as Inconel, a brand for a family of austenitic nickel-chromium-based superalloys from Special Metals Corporation. Advantageously, the second material comprises a paramagnetic material. Particularly advantageously, the second material comprises a non-magnetically susceptible material. In the embodiment illustrated, the first material comprises 304 stainless steel and the second material comprises Inconel. Use of these materials results in the first portion acting as a passive marker when using MRI to create a magnetic resonance image.

While the cannula 12 has been illustrated as including a distal tapered tip 28 with a cutting edge 30, a cannula can include any suitable structural configuration, such as those with blunted, or non-tapered, distal tips. While the cannula 12 has been illustrated as including a first portion 32 that extends from the distal end 22 toward the proximal end 20 and a second portion 34 that extends from the first portion 32 toward the proximal end 20, a first portion and/or second portion of a cannula can be positioned at any suitable location on a cannula, such as between a proximal end and a distal end of a cannula, such that it extends from a proximal end toward a distal end, such that it extends from a distal end toward a proximal end, such that a second portion extends from a first portion and to a proximal end, and any other location considered suitable for a particular embodiment.

The first inner sheath 14 has a proximal end 40, a distal end 42, and a main body 44 that defines a lumen 46 and a tapered distal tip 48. The second inner sheath 16 has a proximal end 50, a distal end 52, and a main body 54 that defines a lumen 56 and a tapered distal tip 58. The outer sheath 18 has a proximal end 60, a distal end 62, and a main body 64 that defines a lumen 66 and a tapered distal tip 68.

While the cannula 12 has been illustrated as being used with a first inner sheath 14, a second inner sheath 16, and an outer sheath 18 to accomplish sequential dilation, a cannula can be used with any suitable number of inner and/or outer sheaths. Selection of a suitable number of inner sheaths and/or outer sheaths to utilize when completing sequential dilation can be based on the bodily passage within which a sequential dilation is being completed. Examples of numbers of inner sheaths and/or outer sheaths considered suitable to include when completing sequential dilation include one, two, a plurality, three, four, more than four, and any other number considered suitable for a particular embodiment.

A cannula, an inner sheath, and an outer sheath can be formed of any suitable MRI compatible material and selection of a suitable material can be based on various considerations, including the intended use of the cannula, inner sheath, and/or outer sheath. Examples of MRI compatible materials considered suitable to form a cannula include biocompatible materials, materials that can be made biocompatible, metals, electrically insulating materials, electrically non-conducting materials, shape memory alloys, including nickel-titanium alloys such as Nitinol, stainless steel, including Austenitic stainless steel, stainless steel containing Iron, stainless steel including Inconel, cobalt chromium, cobalt chromium alloys, Inconel, titanium, polymers, PEEK, carbon-filled PEEK, ceramics, the materials described herein, combinations of the described herein, and any other material considered suitable for a particular embodiment. Examples of materials considered suitable to form an inner sheath and/or an outer sheath include biocompatible materials, materials that can be made biocompatible, metals, electrically insulating materials, electrically non-conducting materials, shape memory alloys, including nickel-titanium alloys such as Nitinol, Inconel, plastics, polymers, PEEK, carbon-filled PEEK, polyethylene, such as high-density polyethylene (HDPE), polypropylene, polycarbonates, silicone, Delrin, ceramics, transparent materials, opaque materials, the materials described herein, combinations of the materials described herein, and any other material considered suitable for a particular embodiment.

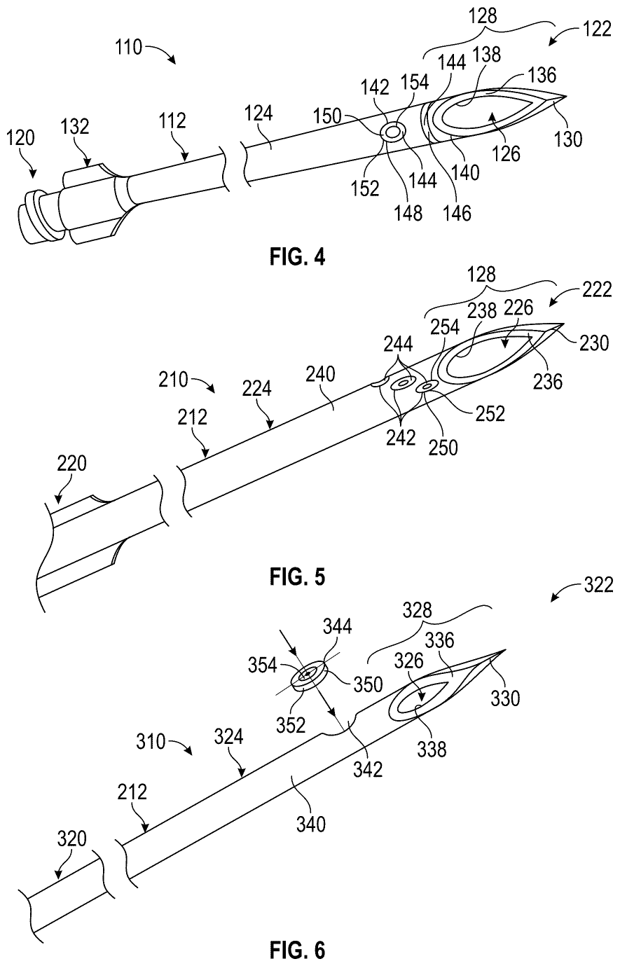

FIG. 4 illustrates a second example medical device 110. In this example, the medical device 110 is a cannula 112. The cannula 112 is similar to the cannula 12 illustrated in FIGS. 1, 2, and 3 and described above, except as detailed below. In the illustrated embodiment, the cannula 112 has a proximal end 120, a distal end 122, and a main body 124 that defines a lumen 126 and a distal tapered tip 128 with a cutting edge 130. The main body 124 is formed of a nickel-chromium based alloy, such as an Inconel brand alloy. The lumen 126 extends from the proximal end 120 to the distal end 122 such that one or more devices can be passed into, and through, the cannula 112. A hub member 132 is disposed on the proximal end 120 of the cannula 112 such that other devices can be attached to the cannula 112.

In the illustrated embodiment, the main body 124 of the cannula 112 defines a wall 136 that has inner 138 and outer 140 opposing surfaces. A wall thickness extends between the inner 138 and outer 140 surfaces. The main body 124 of the cannula 112 defines a passageway 142 that extends through the entire thickness of the wall 136 and from the inner surface 138 to the outer surface 140. Alternatively, a passageway can extend only partially through the thickness of a wall, either from an inner surface toward an outer surface or from an outer surface toward an inner surface.

The cannula 112 includes a plurality of markers 144. In the illustrated embodiment, a first marker 146 of the plurality of makers 144 is disposed between the proximal end 120 and the distal end 122 of the cannula 112 and a second marker 148 of the plurality of markers 144 is disposed between the first marker 146 and the proximal end 120 of the cannula 112. Each marker of the plurality of markers 144 is attached to the cannula 112 and is formed of a magnetically susceptible material, such as a paramagnetic material or a ferromagnetic material. The first marker 146 is disposed circumferentially about main body 124 of the cannula 112 and comprises a stainless steel marker band. The second marker 148 is a plug 150 disposed within the passageway 142. The plug 150 is formed of stainless steel and has a main body 152 that defines a passageway 154 that extends through the plug 150. Alternative embodiments, however, can include a plug that does not define a passageway that extends through the plug such that the plug is a solid member that prevents access to a lumen defined by a cannula. The passageway 154 is in fluid communication with the lumen 126 defined by the cannula 112. The second marker 148 is flush with the inner surface 138 and the outer surface 140 and, in the embodiment illustrated, has been pressed into the passageway 142. Additional securement can be used, if desired. For example, a marker can be laser welded to a wall of a cannula to secure the marker in a passageway. Forming the first and second markers 146, 148 of stainless steel results in the cannula 112 that has two passive markers that can be viewed when using MRI to create a magnetic resonance image. Each marker of the plurality of markers 144 has properties that produce visual artifacts during MRI procedures in which the medical device 110 is imaged. These visual artifacts can be used to determine placement of the medical device 110 relative to other portions of an MRI image, such as portions of a bodily passage into which the medical device 110 has been advanced.

A passageway can be created in a cannula using any suitable technique, such as laser drilling, or electric discharge machining (EDM). A marker included on a cannula can be attached to the cannula using any suitable technique and be formed of any suitable material. Examples of techniques considered suitable to attach a marker to a cannula include pressing, welding, using adhesives, and any other technique considered suitable for a particular embodiment. Examples of materials considered suitable to form a marker include biocompatible materials, materials that can be made biocompatible, MRI compatible materials, metals, electrically insulating materials, electrically non-conducting materials, magnetically susceptible materials, including paramagnetic and ferromagnetic materials, ferromagnetic passive materials, Ferritic Stainless Steel, Ferritic Stainless Steel 430L powder, shape memory alloys, including nickel-titanium alloys such as Nitinol, stainless steel, including Austenitic stainless steel, stainless steel containing Iron, stainless steel including Inconel, cobalt chromium, cobalt chromium alloys, Inconel, titanium, materials (e.g., Stainless Steel) having a hardness of about 192 KSI, the materials described herein, combinations of the described herein, and any other material considered suitable for a particular embodiment. While the first marker 146 has been illustrated as a marker band (e.g., circumferential marker) and the second marker 148 has been illustrated as a plug, a marker can comprise any suitable structure attached to a cannula or any suitable treatment imparted on a cannula. For example, a marker can include bands of material, magnetic inks, sputtered magnetite marks of varying shapes and/or configurations, and/or dimpling or peening the material that forms a cannula (e.g., annealed 304 stainless steel). Any suitable marker can be included in a medical device, such as those described herein. Examples of markers considered suitable to include in a medical device are described in U.S. patent application Ser. No. 16/454,905, filed on Jun. 27, 2019, which is hereby incorporated by reference in its entirety for the purpose of describing markers considered suitable to include in a medical device. Each marker of the plurality of markers 144 can have any suitable configuration. Circumferential markers can be disposed around the entire circumference of the main body 124 of the medical device 110. Alternatively, a marker that defines a partial circumference can be disposed around a main body of a medical device according to an embodiment such that the marker extends around only a portion of the circumference of the main body.

Any suitable number of markers can be used in a medical device according to an embodiment. While the medical device 110 includes two markers 146, 148, it is to be appreciated that a medical device according to an embodiment can include any number of markers considered suitable for the intended use of the particular medical device. Examples of suitable numbers of markers for inclusion in a medical device according to an embodiment include one marker, two markers, more than two markers, three markers, a plurality of markers, four markers, five markers, six markers, seven markers, eight markers, nine markers, ten markers, and more than ten markers. Furthermore, in embodiments that include two or more markers, the markers can be spaced from each other by a desired distance. It should be noted, though, that, because the markers produce visual artifacts and their utility in the medical device is based on this production of visual artifacts in MRI procedures, it is desirable to space markers from each other by a distance that does not result in overlapping or nearly overlapping visual artifacts.

FIG. 5 illustrates a third example medical device 210. In this example, the medical device 210 is a cannula 212. The cannula 212 is similar to the cannula 112 illustrated in FIG. 4 and described above, except as detailed below. In the illustrated embodiment, the cannula 212 has a proximal end 220, a distal end 222, and a main body 224 that defines a lumen 226 and a distal tapered tip 228 with a cutting edge 230. The lumen 226 extends from the proximal end 220 to the distal end 222 such that one or more devices can be passed into, and through, the cannula 212.

In the illustrated embodiment, the main body 224 of the cannula 212 defines a wall 236 that has inner 238 and outer 240 opposing surfaces. The main body 224 of the cannula 212 defines a plurality of passageways 242. Each passageway of the plurality of passageways extends through the entire thickness of the wall 236 and from the inner surface 238 to the outer surface 240.

The cannula 212 includes a plurality of markers 244. In the illustrated embodiment, the plurality of markers 244 is arranged in a helical configuration about the circumference of the cannula 212. Each marker of the plurality of markers 244 is attached to the cannula 212 and comprises a plug 250 disposed within a passageway of the plurality of passageways 242. Each plug 250 has a main body 252 that defines a passageway 254 that extends through the plug 250. The passageway 254 is in fluid communication with the lumen 226 defined by the cannula 212.

FIG. 6 illustrates a fourth example medical device 310. In this example, the medical device 310 is a cannula 312. The cannula 312 is similar to the cannula 312 illustrated in FIG. 4 and described above, except as detailed below. In the illustrated embodiment, the cannula 312 has a proximal end 320, a distal end 322, and a main body 324 that defines a lumen 326 and a distal tapered tip 328 with a cutting edge 330. The lumen 326 extends from the proximal end 320 to the distal end 322 such that one or more devices can be passed into, and through, the cannula 312.

In the illustrated embodiment, the main body 324 of the cannula 312 defines a wall 336 that has inner 338 and outer 340 opposing surfaces. The main body 324 of the cannula 312 defines a passageway 342 that extends through the entire thickness of the wall 336 and from the inner surface 338 to the outer surface 340.

The cannula 312 includes a marker 344. In the illustrated embodiment, the marker 344 is a plug 350 disposed within the passageway 342. The plug 350 has a main body 352 that defines a passageway 354 that extends through the plug 350. The passageway 354 is in fluid communication with the lumen 326 defined by the cannula 312. As such, the marker 344 is attached to the cannula 312.

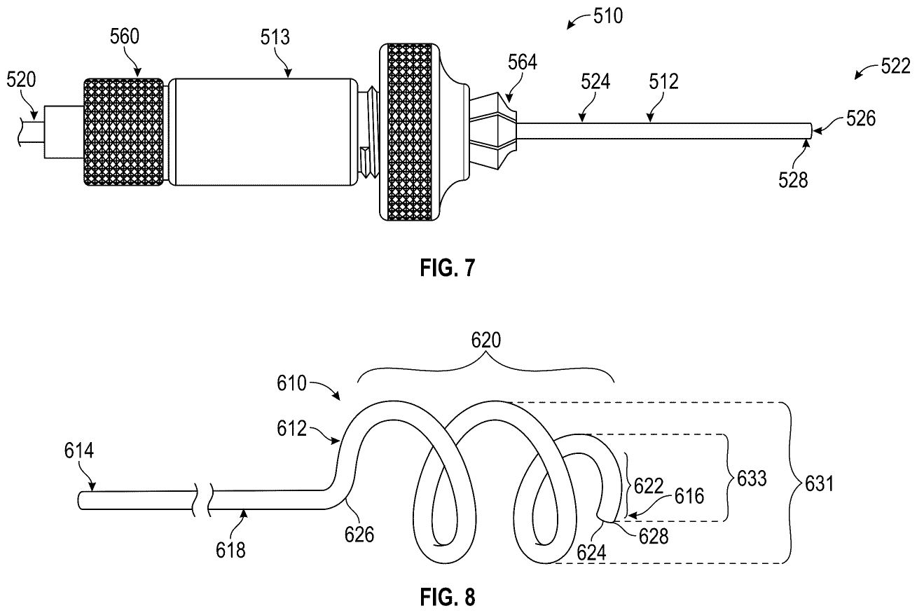

FIG. 7 illustrates a fifth example medical device 510. In this example, the medical device 510 is a cannula 512 with an attached handle 513. The cannula 512 is similar to the cannula 12 illustrated in FIGS. 1, 2, and 3 and described above, except as detailed below. In the illustrated embodiment, the cannula 512 has a proximal end 520, a distal end 522, and a main body 524 that defines a lumen 526 and a blunted distal tip 528. The lumen 526 extends from the proximal end 520 to the distal end 522 such that one or more devices can be passed into, and through, the cannula 512. The blunted distal tip 528 is created by grinding a non-cutting atraumatic tip on the distal end 522 of the cannula 512.

In the illustrated embodiment, the handle 513 is releasably attached to the cannula 512 and comprises a luer-lock hub 560 that includes sealing members and a clamping member 564. The sealing members can be adjusted by rotating the cap of the luer-lock hub 560. The clamping member 564 is moveable between a first configuration and a second configuration. In the first configuration, the clamping member 564 attaches the luer-lock hub 560 to the cannula 512. In the second configuration, the clamping member 564 is free of attachment to the cannula 512 such that the luer-lock hub 560 can be moved along the axial length of the cannula 512. While a male luer-lock hub has been illustrated, alternative embodiments can include a female luer-lock hub. A handle, such as handle 513, can be included on any medical device described herein.

FIG. 8 illustrates a sixth example medical device 610. In this example, the medical device 610 is an anchor member 612. The anchor member 612 has a proximal end 614, a distal end 616, and a main body 618 that defines a coil 620 and a distal tapered tip 622 with a cutting edge 624.

In the illustrated embodiment, the coil 620 has a coil first end 626, a coil second end 628, and defines a plurality of coil turns that creates a conical coil. The coil has a first outside diameter 631 and a second outside diameter 633 that is less than the first outside diameter 631. In the illustrated embodiment, the coil outside diameter tapers from the coil first end 626 to the coil second end 628.

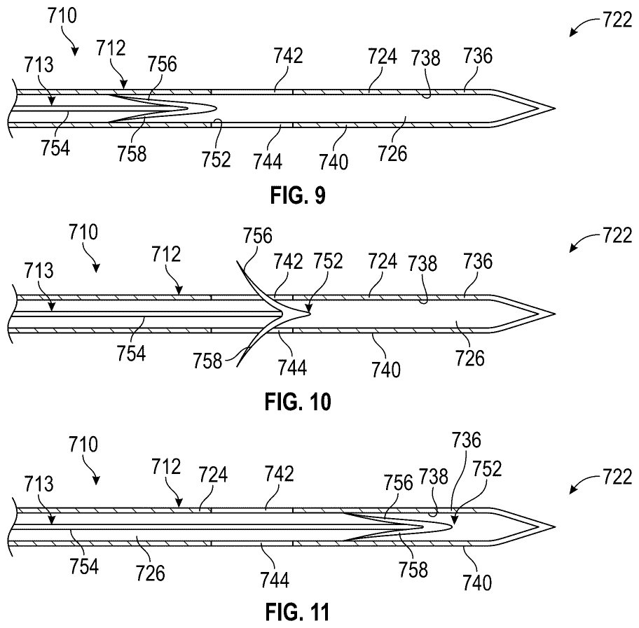

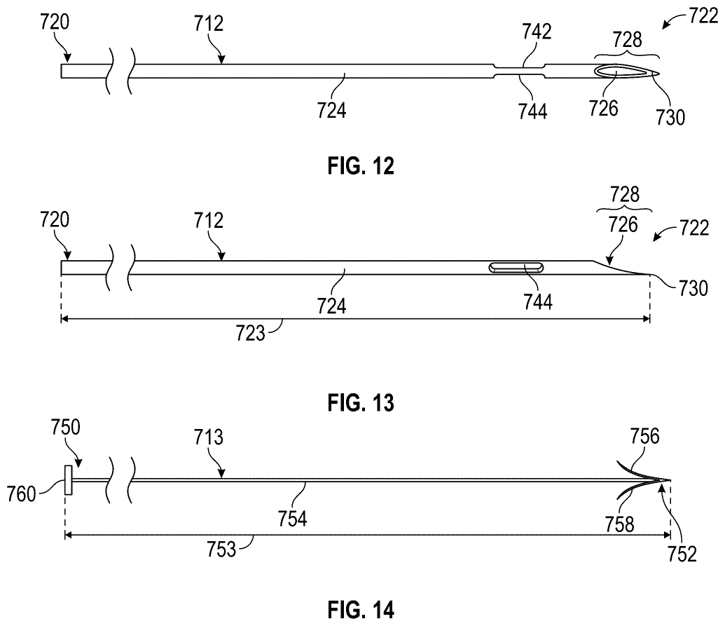

FIGS. 9, 10, 11, 12, 13, and 14 illustrate a seventh example medical device 710. In this example, the medical device 710 includes a cannula 712 and an anchor member 713. The cannula 712 is similar to the cannula 12 illustrated in FIGS. 1, 2, and 3 and described above, except as detailed below. In the illustrated embodiment, the cannula 712 has a proximal end 720, a distal end 722, a length 723, and a main body 724 that defines a lumen 726, and a distal tapered tip 728 with a cutting edge 730. The lumen 726 extends from the proximal end 720 to the distal end 722 such that one or more devices can be passed into, and through, the cannula 712.

In the illustrated embodiment, the main body 724 of the cannula 712 defines a wall 736 that has inner 738 and outer 740 opposing surfaces. The main body 724 of the cannula 712 defines a first passageway 742 and a second passageway 744. Each of the first passageway 742 and the second passageway 744 extends through the entire thickness of the wall 736, from the inner surface 738 to the outer surface 740, and has an oblong shape.

In the illustrated embodiment, the anchor member 713 is partially disposed within the lumen 726 defined by the cannula 712 and has a proximal end 750, a distal end 752, a length 753, an elongate main body 754, a first barb 756, a second barb 758, and a mechanical stop 760. The length 753 of the anchor member is less than the length 723 of the cannula 712. Each of the first barb 756 and the second barb 758 extends from the distal end 752 toward the proximal end 750 and away from the elongate main body 754. The mechanical stop 760 is disposed on the proximal end 750 of the anchor member 713 and provides a mechanism for preventing the anchor member 713 from being advanced beyond the distal end 722 of the cannula 712.

As shown in FIGS. 9, 10, and 11, the anchor member 713 is moveable within the cannula 712 such that it can be advanced into and withdrawn from the lumen 726 defined by the cannula 712. FIG. 9 shows the anchor member 713 in a first position in which the barbs 756, 758 are disposed within the lumen 726 defined by the cannula 712 and between the passageways 742, 744 and the proximal end 720 of the cannula 712. FIG. 10 shows the anchor member 713 in a second position in which the first barb 756 is disposed through the first passageway 742 and the second barb 758 is disposed through the second passageway 744. When disposed within a bodily passage, the second configuration anchors the anchor member 713 within tissue such that the cannula 712 can be utilized to track additional medical devices to a point of treatment. FIG. 11 shows the anchor member 713 in a third position in which the barbs 756, 758 are disposed between the passageways 742, 744 and the distal end 722 of the cannula 712. In use, to move the anchor member 713 from the first position to the second position, a distally-directed force is applied to the anchor member 713 while maintaining the position of the cannula 712. Alternatively, the position of the anchor member 713 can be maintained while applying a proximally-directed force on the cannula 712 or a distally-directed force can be applied to the anchor member 713 while applying a proximally-directed force on the cannula 712. To move the anchor member 713 from the second position to the third position, a distally-directed force is applied to the anchor member 713 while maintaining the position of the cannula 712. Alternatively, the position of the anchor member 713 can be maintained while applying a proximally-directed force on the cannula 712 or a distally-directed force can be applied to the anchor member 713 while applying a proximally-directed force on the cannula 712.

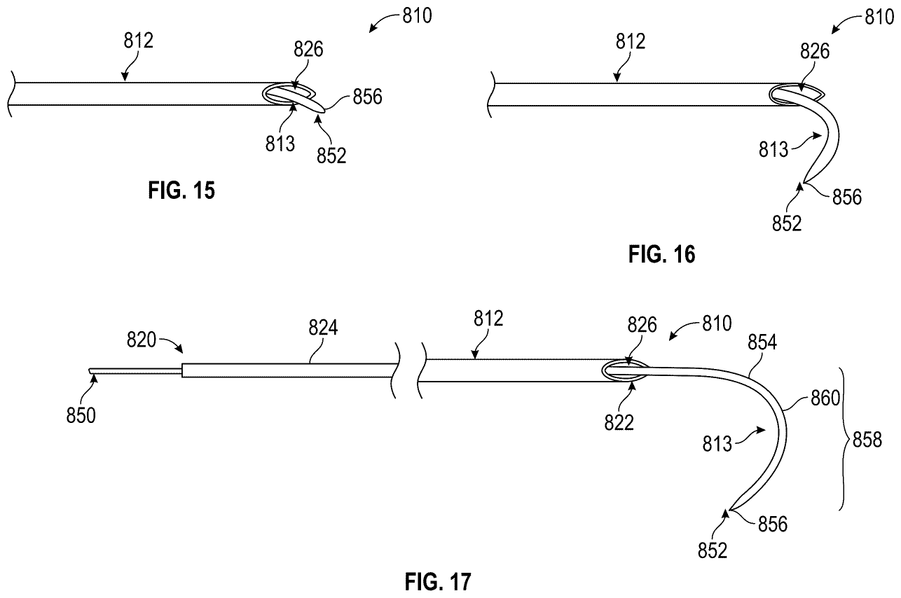

FIGS. 15, 16, and 17 illustrate an eighth example medical device 810. In this example, the medical device 810 includes a cannula 812 and an anchor member 813 disposed within the cannula 812. The cannula 812 is similar to the cannula 12 illustrated in FIGS. 1, 2, and 3 and described above, except as detailed below. In the illustrated embodiment, the cannula 812 has a proximal end 820, a distal end 822, and a main body 824 that defines a lumen 826. The lumen 826 extends from the proximal end 820 to the distal end 822 such that one or more devices can be passed into, and through, the cannula 812.

In the illustrated embodiment, the anchor member 813 has a proximal end 850, a distal end 852, an elongate main body 854, and a distal tapered tip 856. A distal portion 858 of the elongate main body 854 is biased to a curved configuration, as shown in FIG. 17, such that it defines a curve 860 when free of any structures (e.g., cannula 812) or any outside forces. The distal portion 858 of the elongate main body 854 has a substantially straight configuration when disposed within the lumen 826 defined by the cannula 812.

As shown in FIGS. 15, 16, and 17, the anchor member 813 is moveable within the cannula 812 such that it can be advanced into and withdrawn from the lumen 826 defined by the cannula 812. FIG. 15 shows the anchor member 813 in a first position in which the distal tapered tip 856 is disposed outside of the lumen 826 defined by the cannula 812 and the distal portion 858 begins to define the curve 860 relative to the cannula 812. FIG. 16 shows the anchor member 813 in a second position in which the distal tapered tip 856 is disposed outside of the lumen 826 defined by the cannula 812 and the distal portion 858 defines a larger portion of the curve 860 relative to the portion of the curve 860 defined in the first position. FIG. 17 shows the anchor member 813 in a third position in which the distal tapered tip 856 is disposed outside of the lumen 826 defined by the cannula 812 and the distal portion 858 defines the entire curve 860 outside of the lumen 826 defined by the cannula 812. When the anchor member 813 is in the third configuration, the anchor member 813 is anchored within tissue such that the cannula 812 can be utilized to track additional medical devices to a point of treatment. In use, to move the anchor member 813 from the first position to the second position, and from the second position to the third position, a distally-directed force is applied to the anchor member 813 while maintaining the position of the cannula 812. Alternatively, the position of the anchor member 813 can be maintained while applying a proximally-directed force on the cannula 812 or a distally-directed force can be applied to the anchor member 813 while applying a proximally-directed force on the cannula 812.

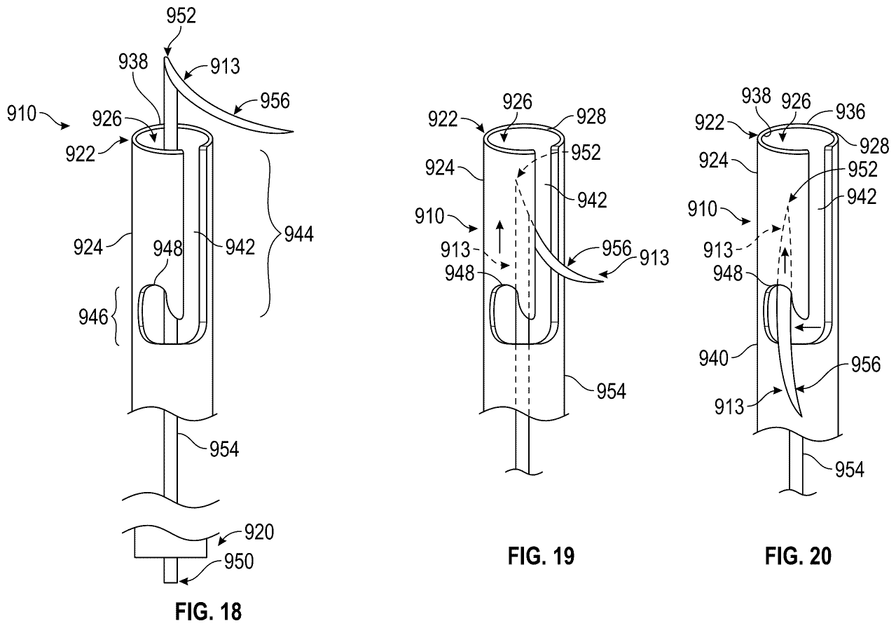

FIGS. 18, 19, and 20 illustrate a ninth example medical device 910. In this example, the medical device 910 is a cannula 912 and an anchor member 913. The cannula 912 is similar to the cannula 12 illustrated in FIGS. 1, 2, and 3 and described above, except as detailed below. In the illustrated embodiment, the cannula 912 has a proximal end 920, a distal end 922, and a main body 924 that defines a lumen 926, and a blunted distal tip 928. The lumen 926 extends from the proximal end 920 to the distal end 922 such that one or more devices can be passed into, and through, the cannula 912.

In the illustrated embodiment, the main body 924 of the cannula 912 defines a wall 936 that has inner 938 and outer 940 opposing surfaces. The main body 924 of the cannula 912 defines a slot 942 that has a first portion 944 and a second portion 946. The slot 942 extends through the entire thickness of the wall 936 and from the inner surface 938 to the outer surface 940. The first portion 944 of the slot 942 extends from the distal end 922 of the cannula 912 and toward the proximal end 920 of the cannula 912. The second portion 946 of the slot 942 extends from the first portion 944 of the slot 942 around a portion of the circumference of the wall 936 of the cannula 912 and toward the distal end 922 of the cannula 912 to a terminal end 948. The first portion 944 and the second portion 946 of the slot 942 cooperatively define a j-shaped slot 942 that can receive a portion of the anchor member 913, as described in more detail herein.

In the illustrated embodiment, the anchor member 913 has a proximal end 950, a distal end 952, an elongate main body 954, and a barb 956. The barb 956 extends from the distal end 952 toward the proximal end 950 and away from the elongate main body 954.

As shown in FIGS. 18, 19, and 20, the anchor member 913 is moveable within the cannula 912 such that it can be advanced into and withdrawn from the lumen 926 defined by the cannula 912. FIG. 18 shows the anchor member 913 in a first position in which the barb 756 is disposed outside of the lumen 926 defined by the cannula 912 and distal to the distal end 922 of the cannula 912. This configuration anchors the anchor member 913 within tissue such that the cannula 912 can be utilized to track additional medical devices to a point of treatment. Positioning the anchor member 913 in the first position can be accomplished by applying a distally-directed force on the anchor member 913 while maintaining the position of the cannula 912. Alternatively, the position of the anchor member 913 can be maintained while applying a proximally-directed force on the cannula 912 or a distally-directed force can be applied to the anchor member 913 while applying a proximally-directed force on the cannula 912. FIG. 19 shows the anchor member 913 in a second position in which the barb 956 is partially disposed within the first portion 944 of the slot 942 defined by the cannula 912. To move the anchor member 913 from the first position to the second position, a distally-directed force is applied on the cannula 913 while maintaining the position of the anchor member 913. Optionally, depending on the orientation of the cannula 912 relative to the anchor member 913, torque can be applied to the cannula 912 to position the slot in axial alignment with the barb 956. FIG. 20 shows the anchor member 913 in a third position in which the barb 956 is partially disposed within the second portion 946 of the slot 942 defined by the cannula 912. To move the anchor member 913 from the second position to the third position, torque is applied to the cannula 913 while maintaining the position of the anchor member 913 to move the barb 956 from the first portion 944 of the slot 942 to the second portion 946 of the slot 942. To fully advance the barb 956 into the lumen 926 defined by the cannula 912, a proximally-directed force is applied to the cannula 912 while maintaining the position of the anchor member 913 and the barb 956 is positioned the second portion 946 of the slot 942 until the barb 956 is disposed within the lumen 926 defined by the cannula 912. Alternatively, to fully advance the barb 956 into the lumen 926 defined by the cannula 912, while the barb 956 is positioned the second portion 946 of the slot 942 a distally-directed force can be applied to the anchor member 913 while maintaining the position of the cannula 912 or a distally-directed force on the anchor member 913 while applying a proximally-directed force on the cannula 912 until the barb 956 is disposed within the lumen 926 defined by the cannula 912.

Various methods of performing interventional medical treatment under MRI are described herein. While the methods described herein are shown and described as a series of acts, it is to be understood and appreciated that the methods are not limited by the order of acts, as some acts may in accordance with these methods may be omitted, occur in the order shown and/or described, occur in different orders, and/or occur concurrently with other acts described herein.

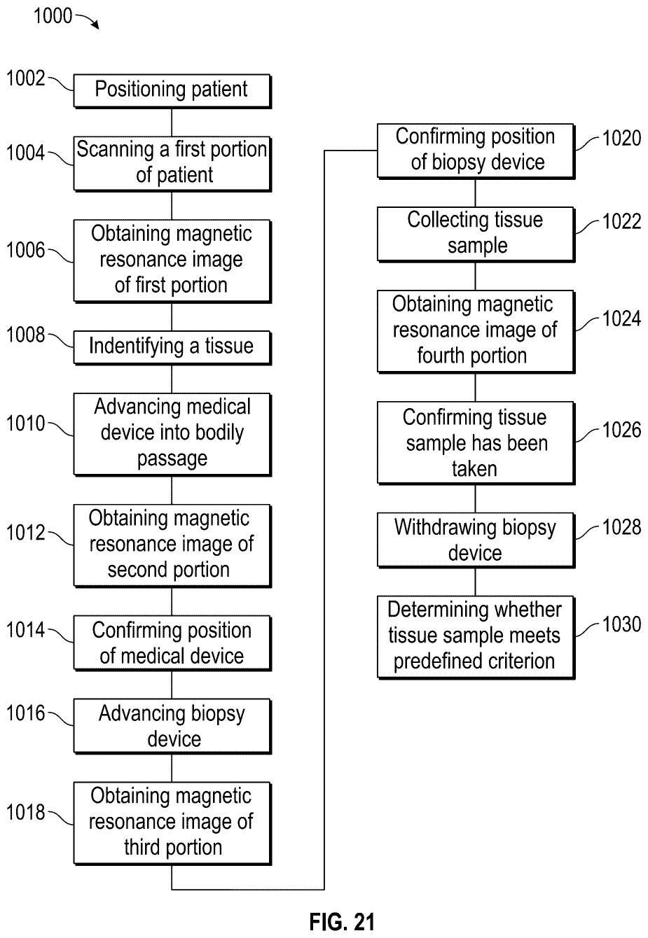

FIG. 21 illustrates a schematic illustration of an example method 1000 of performing treatment under MRI.

An initial step 1002 comprises positioning a patient within a magnetic resonance scanner. Another step 1004 comprises scanning a first portion of the patient using the magnetic resonance scanner. Another step 1006 comprises obtaining a magnetic resonance image of the first portion of the patient. Another step 1008 comprises identifying a tissue that has predefined characteristics using the magnetic resonance image. While the patient remains positioned within the magnetic resonance scanner used to scan a portion of the patient, another step 1010 comprises advancing a medical device into a bodily passage and to the tissue while scanning a second portion of the patient that includes the medical device using the magnetic resonance scanner. Another step 1012 comprises obtaining a magnetic resonance image of the second portion of the patient that includes the medical device. Another step 1014 comprises confirming the position of the medical device within the bodily passage. Another step 1016 comprises advancing a biopsy device through the medical device and to the tissue while scanning a third portion of the patient that includes the biopsy device using the magnetic resonance scanner. Another step 1018 comprises obtaining a magnetic resonance image of the third portion of the patient that includes the biopsy device. Another step 1020 comprises confirming the position of the biopsy device. Another step 1022 comprises collecting a tissue sample from the tissue using the biopsy device while scanning a fourth portion of the patient that includes the biopsy device and the tissue using the magnetic resonance scanner. Another step 1024 comprises obtaining a magnetic resonance image of the fourth portion of the patient that includes the biopsy device. Another step 1026 comprises confirming the tissue sample has been collected. Another step 1028 comprises withdrawing the biopsy device and the tissue sample through the medical device. Another step 1030 comprises determining whether the tissue sample meets a predefined criterion.

Step 1002 can be accomplished by positioning a patient within any suitable magnetic resonance scanner, such as conventional magnetic resonance scanners, magnetic resonance scanners that utilize 0.55 T fields, 1.5 T fields, 3 T fields, fields between about 0.055 T and 1.5 T, fields less than 1 T, and any other magnetic resonance scanner considered suitable for a particular embodiment.

Step 1004 can be accomplished by scanning any suitable portion of a patient and selection of a suitable portion of a patient to scan can be based on various considerations, including the treatment intended to be performed. Examples of portions of a patient considered suitable to scan include the extremities (e.g., arms, legs), chest, breast, spine, neck, head, abdomen, pelvis, prostate, peri-prostatic structures, tissues surrounding the portions of a patient described herein, and/or any other portion of the patient considered suitable for a particular embodiment.

Step 1006 can be accomplished by obtaining the magnetic resonance image from the magnetic resonance scanner used in step 1002.

Step 1008 can be accomplished by reviewing the magnetic resonance image obtained in step 1006 and utilizing conventional techniques and/or methods to determine whether tissue has predefined characteristics (e.g., tissue has characteristics indicative of cancer, is a lesion, abnormal mass). Furthermore, the margins of any tissue (e.g., abnormal mass) can be identified and used in further steps, as described herein, to remove and/or treat the tissue.

Step 1010 can be accomplished using any suitable medical device, such as the medical devices and/or cannulas described herein, or incorporated by reference. Step 1010 can be accomplished by applying a distally-directed force on the medical device such that a distal end of the medical device is advanced into a bodily passage and to, within, or adjacent to, the tissue that has been identified as having the predefined characteristics. A bodily passage can include any suitable portion of a body, including existing bodily passages, bodily lumens, and/or bodily passages created through tissues layers and/or fascia using a device described herein. Step 1010 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the medical device, portions that include the tissue that has predefined characteristics and the medical device, and any other portion of the patient considered suitable for a particular embodiment. For example, a second portion of the patient can be the same as, or different than, the first portion of the patient.

Step 1012 can be accomplished by obtaining the magnetic resonance image from the magnetic resonance scanner used in step 1002.

Step 1014 can be accomplished by reviewing the magnetic resonance image obtained in step 1012 and confirming the medical device is positioned at a desired location within the bodily passage (e.g., at, within, or adjacent to, the tissue that has been identified as having the predefined characteristics). This can be accomplished by visualizing one or more markers included on the medical device, as described herein. If the medical device is not positioned at a desired location, an optional step comprises manipulating the position of the medical device.

Step 1016 can be accomplished by applying a distally-directed force on the biopsy device such that a distal end of the biopsy device is advanced into a lumen defined by the medical device, through the lumen defined by the medical device, and to the tissue that has been identified as having the predefined characteristics. Step 1016 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a third portion of the patient can be the same as, or different than, the first portion of the patient and/or the second portion of the patient.

Step 1016, step 1022, and step 1028 can be accomplished using any suitable biopsy device, such as MRI compatible biopsy devices, the Echotip ProCore provided by Cook Medical, the Echotip Ultra provided by Cook Medical, and any other biopsy device considered suitable for a particular embodiment. Alternatively, step 1016, step 1022, and step 1028 can be completed using a biopsy device that is not MRI compatible. This alternative step can comprise advancing the biopsy device through the medical device and to the tissue without scanning a portion of the patient that includes the biopsy device using the magnetic resonance scanner. An alternative to step 1028 can comprise collecting a tissue sample using the biopsy device without scanning a portion of the patient that includes the biopsy device and the tissue using the magnetic resonance scanner.

Step 1018 can be accomplished by obtaining the magnetic resonance image from the magnetic resonance scanner used in step 1002.

Step 1020 can be accomplished by reviewing the magnetic resonance image obtained in step 1018 and confirming the biopsy device is positioned at a desired location within the bodily passage (.e.g., at, within, or adjacent to, the tissue that has been identified as having the predefined characteristics). If the biopsy device is not positioned at a desired location, an optional step comprises manipulating the position of the biopsy device.

Step 1022 can be accomplished using the biopsy device and conventional methods of obtaining a sample using a biopsy device. Step 1022 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a fourth portion of the patient can be the same as, or different than, the first portion of the patient, the second portion of the patient, and/or the third portion of the patient.

Step 1010, step 1016, and/or step 1022 can optionally be conducted in combination with performing an ultrasound on the portion of the patient that includes the medical device and/or biopsy device. In embodiments in which an ultrasound image is obtained, the magnetic resonance image obtained can be electronically fused with a real-time ultrasound image (e.g., transrectal ultrasound image of a prostate).

Step 1024 can be accomplished by obtaining the magnetic resonance image from the magnetic resonance scanner used in step 1002.

Step 1026 can be accomplished by reviewing the magnetic resonance image obtained in step 1024 and confirming the biopsy device has collected the tissue sample (e.g., the tissue that has been identified as having the predefined characteristics). If the biopsy device has not collected the tissue sample, optional steps comprise repeating step 1016, step 1018, step 1020, step 1022, step 1024, and/or step 1026.

Step 1006, step 1012, step 1018, and/or step 1024 can comprise obtaining a single still image. Alternatively, step 1004, step 1010, step 1016, and/or step 1022 can be repeated any desired number of times such that step 1006, step 1012, step 1018, and/or step 1024 comprises obtaining multiple magnetic resonance images of a portion that can be grouped as a cine to show motion.

Step 1028 can be accomplished by applying a proximally-directed force on the biopsy device such that it is withdrawn from the lumen defined by the medical device. In an alternative embodiment, step 1016, step 1018, step 1020, step 1022, step 1024, step 1026, and step 1028 can be omitted from method 1000, and other methods described herein, and the medical device advanced in step 1010 can be utilized to obtain a biopsy and steps similar to those described with respect to a biopsy device can be completed utilizing the medical device. In another alternative embodiment, an anchor can be placed, as described herein, the medical device withdrawn, and the anchor used to track a biopsy device to the tissue.

Step 1030 can be accomplished using any technique or method considered suitable to determine whether tissue meets predefined criterion. For example, step 1030 can utilize conventional techniques and methods for determining whether a tissue sample is malignant, such as frozen section and/or other cytological methods.

Each of step 1006, step 1008, step 1010, step 1012, step 1014, step 1016, step 1018, step 1020, step 1022, step 1024, step 1026, step 1028, and/or step 1030 can be accomplished without removing the patient from the magnetic resonance scanner within which the patient is positioned in step 1002.

Method 1000 is considered advantageous at least because each step of method 1000 can be performed during a single patient visit and using the same magnetic resonance scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a magnetic resonance scanner and other steps have not been described as being performed while scanning a portion of the patient using a magnetic resonance scanner, any step described herein can be completed while scanning a portion of a patient using the magnetic resonance scanner, and/or an ultrasound device or without scanning a portion of a patient using a magnetic resonance scanner. In embodiments in which an ultrasound image is obtained, the magnetic resonance image obtained can be electronically fused with a real-time ultrasound image (e.g., transrectal ultrasound image of a prostate). While some steps have been described as being completed while scanning a portion of the patient using a magnetic resonance scanner, this step can be broken into two separate steps such that a subsequent step of scanning a portion of a patient using the magnetic resonance scanner can be accomplished. Furthermore, any step which is completed while scanning a portion of the patient using the magnetic resonance scanner can comprise obtaining a single still image and be repeated any desired number of times to obtain multiple magnetic resonance images that can be grouped as a cine to show motion and/or any step which is completed while scanning a portion of the patient using the magnetic resonance scanner can comprise obtaining a live image, such as being completed under live real-time MRI visualization.

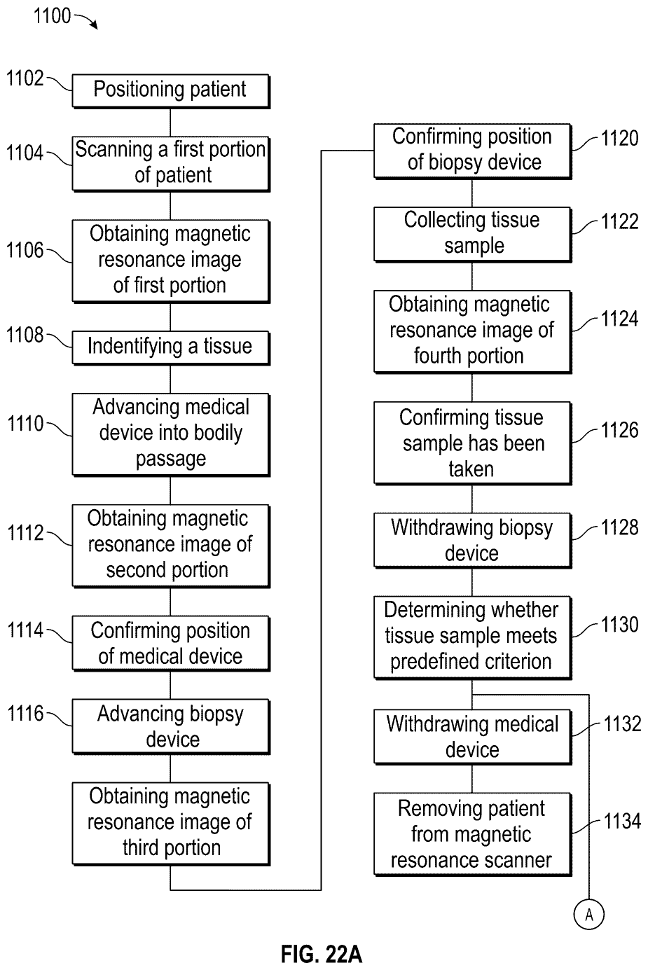

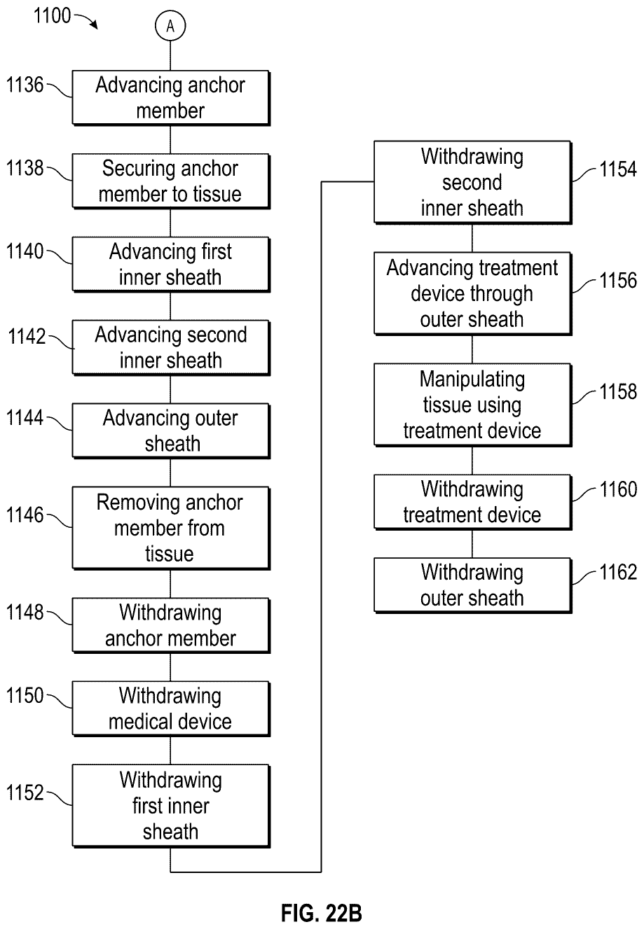

FIGS. 22A and 22B illustrate another schematic illustration of an example method 1100 of performing treatment under MRI.

An initial step 1102 comprises positioning a patient within a magnetic resonance scanner. Another step 1104 comprises scanning a first portion of the patient using the magnetic resonance scanner. Another step 1106 comprises obtaining a magnetic resonance image of the first portion of the patient. Another step 1108 comprises identifying a tissue that has predefined characteristics using the magnetic resonance image. While the patient remains positioned within the magnetic resonance scanner used to scan a portion of the patient, another step 1110 comprises advancing a medical device into a bodily passage and to, within, or adjacent to, the tissue while scanning a second portion of the patient that includes the medical device using the magnetic resonance scanner. Another step 1112 comprises obtaining a magnetic resonance image of the second portion of the patient that includes the medical device. Another step 1114 comprises confirming the position of the medical device within the bodily passage. Another step 1116 comprises advancing a biopsy device through the medical device and to the tissue while scanning a third portion of the patient that includes the biopsy device using the magnetic resonance scanner. Another step 1118 comprises obtaining a magnetic resonance image of the third portion of the patient that includes the biopsy device. Another step 1120 comprises confirming the position of the biopsy device. Another step 1122 comprises collecting a tissue sample from the tissue using the biopsy device while scanning a fourth portion of the patient that includes the biopsy device and the tissue using the magnetic resonance scanner. Another step 1124 comprises obtaining a magnetic resonance image of the fourth portion of the patient that includes the biopsy device. Another step 1126 comprises confirming the tissue sample has been collected. Another step 1128 comprises withdrawing the biopsy device and the tissue sample through the medical device. Another step 1130 comprises determining whether the tissue sample meets a predefined criterion. If the tissue sample does not meet the predefined criterion, additional steps comprise 1132 withdrawing the medical device from the bodily passage and 1134 removing the patient from the magnetic resonance scanner. If the tissue sample meets the predefined criterion, another step 1136 comprises advancing an anchor member through the medical device through which the biopsy device was advanced and to the tissue while the patient remains positioned within the magnetic resonance scanner. Another step 1138 comprises securing the anchor member to the tissue to retain the position of the medical device relative to the tissue. Another step 1140 comprises advancing a first inner sheath over the medical device and toward the tissue to dilate the bodily passage. Another step 1142 comprises advancing a second inner sheath over the first inner sheath and toward the tissue to dilate the bodily passage. Another step 1144 comprises advancing an outer sheath over the second inner sheath and toward the tissue to dilate the bodily passage. Another step 1146 comprises removing the anchor member from the tissue. Another step 1148 comprises withdrawing the anchor member from the bodily passage. Another step 1150 comprises withdrawing the medical device from the bodily passage. Another step 1152 comprises withdrawing the first inner sheath from the bodily passage. Another step 1154 comprises withdrawing the second inner sheath from the bodily passage. Another step 1156 comprises advancing a treatment device through the outer sheath and to the tissue. Another step 1158 comprises manipulating the tissue using the treatment device. Another step 1160 comprises withdrawing the treatment device from the outer sheath. Another step 1162 comprises withdrawing the outer sheath from the bodily passage.

Step 1102 can be completed as described herein with respect to step 1002. Step 1104 can be completed as described herein with respect to step 1004. Step 1106 can be completed as described herein with respect to step 1006. Step 1108 can be completed as described herein with respect to step 1008. Step 1110 can be completed as described herein with respect to step 1010. Step 1112 can be completed as described herein with respect to step 1012. Step 1114 can be completed as described herein with respect to step 1014. Step 1116 can be completed as described herein with respect to step 1016. Step 1118 can be completed as described herein with respect to step 1018. Step 1120 can be completed as described herein with respect to step 1020. Step 1122 can be completed as described herein with respect to step 1022. Step 1124 can be completed as described herein with respect to step 1024. Step 1126 can be completed as described herein with respect to step 1026. Step 1128 can be completed as described herein with respect to step 1028. Step 1130 can be completed as described herein with respect to step 1030.

Step 1132 can be accomplished by applying a proximally-directed force on the medical device until it is withdrawn from the bodily passage. Step 1134 can be accomplished by withdrawing the patient from the magnetic resonance scanner such that the patient is free of the magnetic resonance scanner.

Step 1136 can be accomplished using any suitable anchor member, such as the anchor members described herein. Step 1136 can be accomplished by applying a distally-directed force on the anchor member such that a distal end of the anchor member is advanced into a lumen defined by the medical device, through the lumen defined by the medical device, and to the tissue, within the tissue, or adjacent to the tissue. Optionally, step 1136 can be completed while scanning a fifth portion of the patient that includes the anchor member using the magnetic resonance scanner and additional steps that can be completed subsequent to this optional step include obtaining a magnetic resonance image of the fifth portion of the patient that includes the anchor member, and confirming the position of the anchor member. In embodiments in which it is desired to utilize the magnetic resonance scanner on a second patient that is different from the patient, optional steps that can be completed subsequent to step 1128 include removing the patient from the magnetic resonance scanner while maintaining the position of the patient on a surface, positioning a second patient within the magnetic resonance scanner, scanning a portion of the second patient using the magnetic resonance scanner, obtaining a magnetic resonance image of the portion of the second patient, removing the second patient from the magnetic resonance scanner, and repositioning the patient within the magnetic resonance scanner.

Completion of step 1138 of securing the anchor member into the tissue will depend on the structural configuration of the anchor member. Examples of suitable actions that can be performed for this step include, but are not limited to, axially advancing the anchoring member through a lumen defined by a medical device until a portion of the anchor member (e.g., barb) becomes disposed within the tissue, or applying torque on the anchoring member until a portion of the anchor member becomes disposed within the tissue. Alternatively, step 1138 can comprise anchoring the anchor member into a second, different tissue disposed within or adjacent to the tissue. Step 1138 allows for the medical device to be utilized as a guide rail to a target site such that one or more other devices can be advanced over the medical device to a treatment site. Optionally, step 1138 can be completed while scanning a sixth portion of the patient that includes the anchor member using the magnetic resonance scanner and additional steps that can be completed subsequent to this optional step include obtaining a magnetic resonance image of the sixth portion of the patient that includes the anchor member, and confirming the position of the anchor member. Step 1138 allows for site retention such that the medical device and/or anchor member stays positioned relative to the tissue after biopsy and prior to treatment and can be used to direct a treatment device to the tissue, as described in more detail herein.

Step 1140 can be accomplished by applying a distally-directed force on the first inner sheath such that a distal end of the first inner sheath is advanced over the medical device and to the tissue. Optionally, step 1140 can be completed while scanning a seventh portion of the patient that includes the first inner sheath using the magnetic resonance scanner and additional steps that can be completed subsequent to this optional step include obtaining a magnetic resonance image of the seventh portion of the patient that includes the first inner sheath, and confirming the position of the first inner sheath.

Step 1142 can be accomplished by applying a distally-directed force on the second inner sheath such that a distal end of the second inner sheath is advanced over the first inner sheath and to the tissue. Optionally, step 1142 can be completed while scanning an eighth portion of the patient that includes the second inner sheath using the magnetic resonance scanner and additional steps that can be completed subsequent to this optional step include obtaining a magnetic resonance image of the eighth portion of the patient that includes the second inner sheath, and confirming the position of the second inner sheath.

Step 1144 can be accomplished by applying a distally-directed force on the outer sheath such that a distal end of the outer sheath is advanced over the second inner sheath and to the tissue. Optionally, step 1144 can be completed while scanning a ninth portion of the patient that includes the outer sheath using the magnetic resonance scanner and additional steps that can be completed subsequent to this optional step include obtaining a magnetic resonance image of the ninth portion of the patient that includes the outer sheath, and confirming the position of the outer sheath.

Completion of step 1140, step 1142, and 1144 results in sequential dilation of the bodily passage under MRI. While only first and second inner sheaths and a single outer sheath has been described as accomplishing sequential dilation, any suitable number of inner sheaths and/or outer sheaths can be used to complete sequential dilation of a bodily passage. Examples of numbers of inner sheaths and/or outer sheaths considered suitable to accomplish sequential dilation includes one, two, a plurality, three, four, and any other number considered suitable for a particular embodiment. In embodiment in which sequential dilation is not desired, step 1140 can alternatively comprise advancing a dilator over the medical device and toward the tissue to dilate the bodily passage and step 1142 and step 1144 can be omitted from method 1100. In this alternative step, step 1140 can be accomplished using any suitable dilator, such as conventional dilators, and/or the dilators described herein.

Completion of step 1146 of removing the anchor member from the tissue will depend on the structural configuration of the anchor member. Examples of suitable actions that can be performed for this step include, but are not limited to, applying a proximally-directed force on the anchor member such that it is withdrawn through the lumen defined by a medical device until a portion of the anchor member (e.g., barb) becomes free of the tissue (e.g., in embodiments in which anchor member 813 is being utilized), applying torque to the anchoring member until the portion of the anchor member disposed within the tissue becomes free of the tissue (e.g., in embodiments in which anchor member 612 is being utilized), or applying a distally-directed force on the anchor member such that it is advanced within the lumen defined by a medical device until a portion of the anchor member (e.g., barb) becomes free of the tissue (e.g., in embodiments in which anchor member 713 is being utilized). Any of the steps described herein relative to a particular anchor member can be used to introduce and/or withdraw an anchor member from the tissue. Alternatively, step 1146 can comprise removing the anchor member from a tissue disposed within or adjacent to the tissue. Optionally, step 1146 can be completed while scanning a tenth portion of the patient that includes the anchor member using the magnetic resonance scanner and additional steps that can be completed subsequent to this optional step include obtaining a magnetic resonance image of the tenth portion of the patient that includes the anchor member, and confirming the position of the anchor member.

Step 1148 can be accomplished by applying a proximally-directed force on the anchor member such that it is withdrawn from the lumen defined by the medical device. In an alternative embodiment, a medical device, such as medical device 500, can be advanced through the lumen defined by the dilator (e.g., outer sheath), over the anchor member, and to the tissue such that step 1148 can be completed by applying a proximally-directed force on the anchor member such that it is withdrawn from the lumen defined by the dilator (e.g., outer sheath). In an alternative embodiment, step 1136, step 1138, step 1146, and step 1148 can be omitted from method 1100 and similar steps can be omitted from other methods described herein.

Step 1150 can be accomplished by applying a proximally-directed force on the medical device until it is withdrawn from the bodily passage.

In an alternative embodiment, step 1140 can comprise withdrawing the medical device from the bodily passage, step 1142 can comprise advancing a dilator over the anchor member and toward the tissue to dilate the bodily passage, step 1144 can comprise removing the anchor member from the tissue, and step 1146 can comprise withdrawing the anchor member from the bodily passage. In this alternative embodiment, dilation is accomplished over the anchor member. Sequential dilation can also be accomplished over the anchor member and be completed as described herein utilizing original step 1140, step 1142, and step 1144.

Step 1152 can be accomplished by applying a proximally-directed force on the first inner sheath until it is withdrawn from the bodily passage.

Step 1154 can be accomplished by applying a proximally-directed force on the second inner sheath until it is withdrawn from the bodily passage.

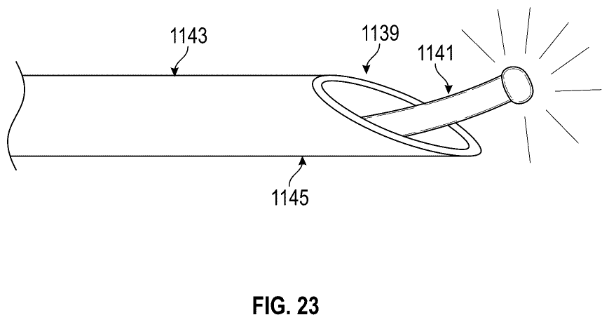

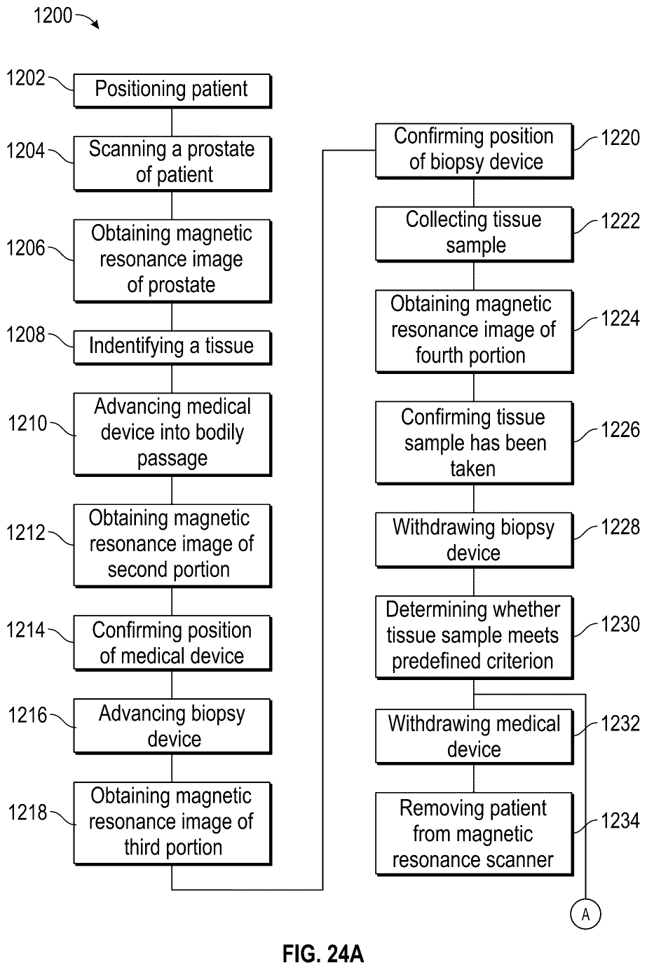

Step 1156 can be accomplished by applying a distally-directed force on the treatment device such that a distal end of the treatment device is advanced into a lumen defined by the outer sheath, or dilator, through the lumen defined by the outer sheath, or dilator, and to the tissue. Any suitable treatment device can be utilized in method 1100 and selection of a suitable treatment device can be based on various considerations, such as the intended use of the treatment device. Examples of treatment devices considered suitable to treat tissue for which a tissue sample meets a predefined criterion include dissection tools, optical fibers, optical fibers formed of a material selected from the group consisting of argon, dye, erbium, excimer, Nd: YAG, and CO2, optical fibers that include control cables (e.g., ultra-high molecular weight polyethylene, Dyneema) to direct the fibers toward tissue intended to be treated, needles, cannulas, such as those described herein or incorporated by reference, and any other treatment device considered suitable for a particular embodiment. FIG. 23 illustrates a tenth example medical device 1139. In the illustrated embodiment, the medical device comprises an optical fiber 1141 that can be utilized to treat tissue disposed within a cannula 1143. The cannula 1143 has a beveled end 1145 that is curved allowing the optical fiber 1141 to exit the cannula 1143 at a 90 degree angle relative to a lengthwise axis of the cannula 1143. Torque can be applied to the optical fiber 1141 and/or cannula 1143 to treat tissue various portions of the tissue. The optical fiber 1141 illustrated in FIG. 23 is configured to transmit a wide range of frequencies or laser light. Optionally, step 1156 can be completed while scanning an eleventh portion of the patient that includes the treatment device using the magnetic resonance scanner and additional steps that can be completed subsequent to this optional step include obtaining a magnetic resonance image of the eleventh portion of the patient that includes the treatment device, and confirming the position of the treatment device.

Step 1158 can be accomplished by physically manipulating the tissue using the treatment device, which can include removing of all or a portion of the tissue, introducing a material into the tissue, applying a treatment to the tissue, and performing any other suitable treatment on the tissue. Examples of treatments considered suitable include laser direct therapy, photodynamic therapy (PDT), chemotherapy, a focal treatment, a radical prostatectomy, infusion of ablative agents, such as acetic acid, ethanol, sclerosants (e.g., sodium tetradecyl sulfate), chemotherapeutic agents, and any other treatment considered suitable for a particular embodiment. Optionally, step 1158 can be completed while scanning a twelfth portion of the patient that includes the treatment device using the magnetic resonance scanner and additional steps that can be completed subsequent to this optional step include obtaining a magnetic resonance image of the twelfth portion of the patient that includes the treatment device, and confirming the position of the treatment device. For example, a magnetic resonance scanner can be utilized to determine areas of tissue that become devitalized during ablative therapies. In embodiments in which one of the cannulas illustrated in FIGS. 1, 4, 5, and 6, or a cannula that includes one or more features of these cannulas, is utilized, treatment can include passing a therapeutic agent (e.g., ablative agent, photosensitizers, and/or nanoparticles) through the lumen defined by the cannula. The first portion and/or the plug(s) included in the cannula provide a mechanism for visualizing the position of these features using a magnetic resonance image such that any infusate can be properly placed within, or adjacent to, the tissue. In addition, this approach improves distribution of any therapeutic agent introduced through the cannula and to a point of treatment.

Step 1160 can be accomplished by applying a proximally-directed force on the treatment device until it is withdrawn from the lumen defined by the outer sheath, or dilator.

Step 1162 can be accomplished by applying a proximally-directed force on the outer sheath, or dilator, until it is withdrawn from the bodily passage.

Method 1100 is considered advantageous at least because each step of method 1100 can be performed during a single patient visit and using the same magnetic resonance scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a magnetic resonance scanner and other steps have not been described as being performed while scanning a portion of the patient using a magnetic resonance scanner, any step described herein can be completed while scanning a portion of a patient using the magnetic resonance scanner and/or an ultrasound device or without scanning a portion of a patient using a magnetic resonance scanner. In embodiments in which an ultrasound image is obtained, the magnetic resonance image obtained can be electronically fused with a real-time ultrasound image (e.g., transrectal ultrasound image of a prostate). Furthermore, any step which is completed while scanning a portion of the patient using the magnetic resonance scanner can comprise obtaining a single still image and be repeated any desired number of times to obtain multiple magnetic resonance images that can be grouped as a cine to show motion and/or any step which is completed while scanning a portion of the patient using the magnetic resonance scanner can comprise obtaining a live image, such as being completed under live real-time MRI visualization.

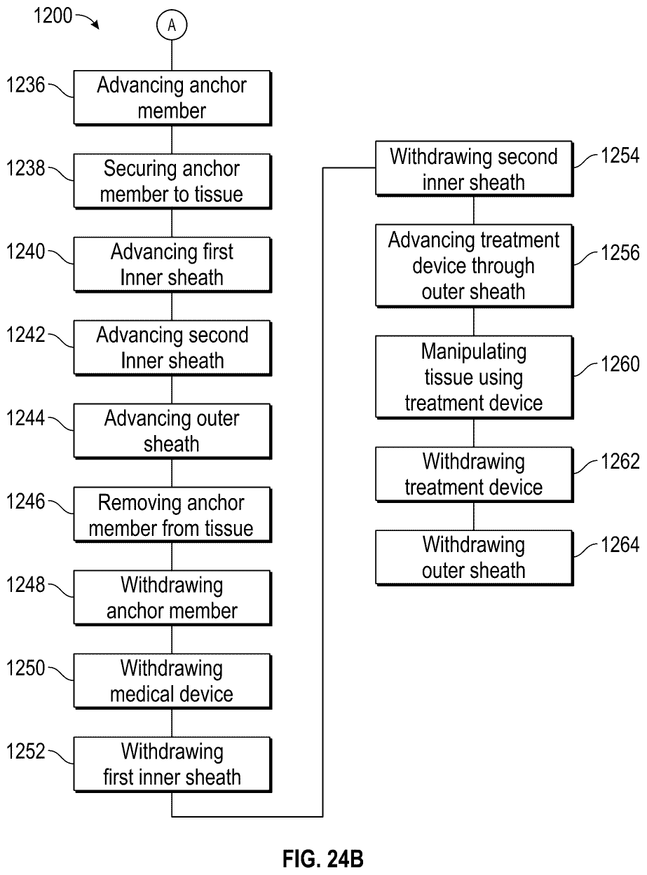

FIGS. 24A and 24B illustrate another schematic illustration of an example method 1200 of performing treatment on a prostate under MRI.

An initial step 1202 comprises positioning a patient within a magnetic resonance scanner. Another step 1204 comprises scanning a prostate and surrounding tissue of the patient using the magnetic resonance scanner. Another step 1206 comprises obtaining a magnetic resonance image of the prostate and surrounding tissue of the patient. Another step 1208 comprises identifying a tissue within the magnetic resonance image that has predefined characteristics. While the patient remains positioned within the magnetic resonance scanner used to scan the prostate and surrounding tissue, another step 1210 comprises advancing a medical device into a bodily passage and to the tissue while scanning a first portion of the patient that includes the medical device using the magnetic resonance scanner. Another step 1212 comprises obtaining a magnetic resonance image of the second portion of the patient that includes the medical device. Another step 1214 comprises confirming the position of the medical device within the bodily passage. Another step 1216 comprises advancing a biopsy device through the medical device and to the tissue while scanning a third portion of the patient that includes the biopsy device using the magnetic resonance scanner. Another step 1218 comprises obtaining a magnetic resonance image of the third portion of the patient that includes the biopsy device. Another step 1220 comprises confirming the position of the biopsy device. Another step 1222 comprises collecting a tissue sample from the tissue using the biopsy device while scanning a fourth portion of the patient that includes the biopsy device and the tissue using the magnetic resonance scanner. Another step 1224 comprises obtaining a magnetic resonance image of the fourth portion of the patient that includes the biopsy device. Another step 1226 comprises confirming the tissue sample has been collected. Another step 1228 comprises withdrawing the biopsy device and the tissue sample through the medical device. Another step 1230 comprises determining whether the tissue sample meets a predefined criterion. If the tissue sample does not meet the predefined criterion, additional steps comprise 1232 withdrawing the medical device from the bodily passage and 1234 removing the patient from the magnetic resonance scanner. If the tissue sample meets the predefined criterion, another step 1236 comprises advancing an anchor member through the medical device through which the biopsy device was advanced and to the tissue while the patient remains positioned within the magnetic resonance scanner. Another step 1238 comprises securing the anchor member to the tissue to retain the position of the medical device relative to the tissue. Another step 1240 comprises advancing a first inner sheath over the medical device and toward the tissue to dilate the bodily passage. Another step 1242 comprises advancing a second inner sheath over the first inner sheath and toward the tissue to dilate the bodily passage. Another step 1244 comprises advancing an outer sheath over the second inner sheath and toward the tissue to dilate the bodily passage. Another step 1246 comprises removing the anchor member from the tissue. Another step 1248 comprises withdrawing the anchor member from the bodily passage. Another step 1250 comprises withdrawing the medical device from the bodily passage. Another step 1252 comprises withdrawing the first inner sheath from the bodily passage. Another step 1254 comprises withdrawing the second inner sheath from the bodily passage. Another step 1256 comprises advancing a treatment device through the outer sheath and to the tissue. Another step 1260 comprises manipulating the tissue using the treatment device. Another step 1262 comprises withdrawing the treatment device from the outer sheath. Another step 1264 comprises withdrawing the outer sheath from the bodily passage.

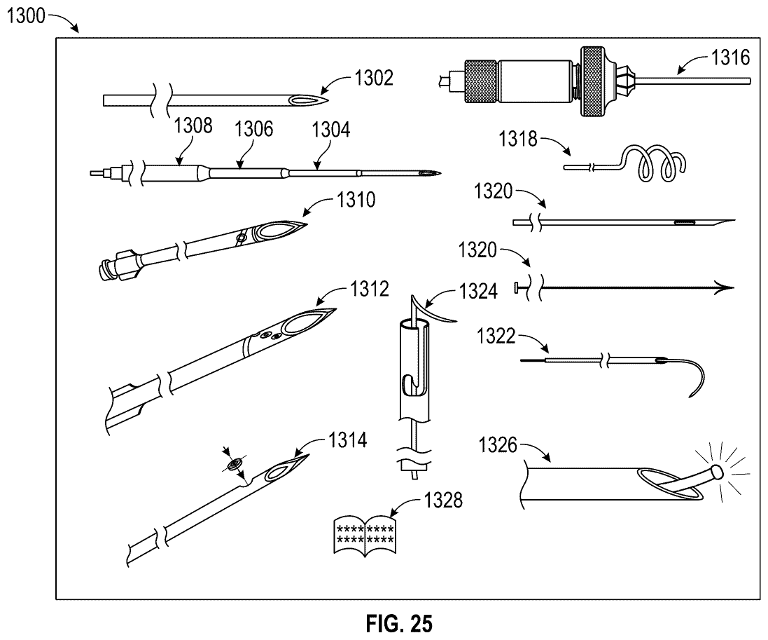

FIG. 25 illustrates an exemplary kit 1300 comprising a first medical device 1302 according to an embodiment, such as medical device 10 illustrated in FIG. 1; a first inner sheath 1304 according to an embodiment, such as first inner sheath 14 illustrated in FIG. 2; a second inner sheath 1306 according to an embodiment, such as second inner sheath 16 illustrated in FIG. 2; an outer sheath 1308 according to an embodiment, such as outer sheath 18 illustrated in FIG. 2; a second medical device 1310 according to an embodiment, such as medical device 110 illustrated in FIG. 4; a third medical device 1312 according to an embodiment, such as medical device 210 illustrated in FIG. 5; a fourth medical device 1314 according to an embodiment, such as medical device 310 illustrated in FIG. 6; a fifth medical device 1316 according to an embodiment, such as medical device 510 illustrated in FIG. 7; a sixth medical device 1318 according to an embodiment, such as medical device 610 illustrated in FIG. 8; a seventh medical device 1320 according to an embodiment, such as medical device 710 illustrated in FIGS. 9, 10, 11, 12, 13, and 14; an eighth medical device 1322 according to an embodiment, such as medical device 810 illustrated in FIGS. 15, 16, and 17; a ninth medical device 1324 according to an embodiment, such as medical device 910 illustrated in FIGS. 18, 19, and 20; a tenth medical device 1326 according to an embodiment, such as medical device 1139 illustrated in FIG. 23; and instructions for use 1328.

While kit 1300 has been illustrated as including ten medical devices 1302, 1310, 1312, 1314, 1316, 1318, 1320, 1322, 1324, 1326, two inner sheaths 1304, 1306, and an outer sheath 1308, any suitable number, and type, of medical devices, inner sheaths, and/or outer sheaths can be included in a kit. Selection of a suitable number of medical devices, inner sheaths, and/or outer sheaths to include in a kit according to a particular embodiment can be based on various considerations, such as the treatment intended to be performed. Examples of numbers of medical devices, inner sheaths, and/or outer sheaths considered suitable to include in a kit include at least one, one, two, a plurality, three, four, five, six, seven, eight, nine, ten, more than ten, and any other number considered suitable for a particular embodiment.

Furthermore, while medical device 10, first inner sheath 14, second inner sheath 16, outer sheath 18, medical device 110, medical device 210, medical device 310, medical device 510, medical device 610, medical device 710, medical device 810, medical device 910, and medical device 1139 have been illustrated as included in kit 1300, any suitable medical device, inner sheath, and/or outer sheath can be included in a kit. Selection of a suitable medical device, inner sheath, and/or outer sheath to include in a kit according to a particular embodiment can be based on various considerations, such as the treatment intended to be performed. Examples of medical devices, inner sheaths, and outer sheaths considered suitable to include in a kit include medical device 10, first inner sheath 14, second inner sheath 16, outer sheath 18, medical device 110, medical device 210, medical device 310, medical device 510, medical device 610, medical device 710, medical device 810, medical device 910, and medical device 1139, and/or any other medical device, inner sheath, and/or outer sheath considered suitable for a particular embodiment.

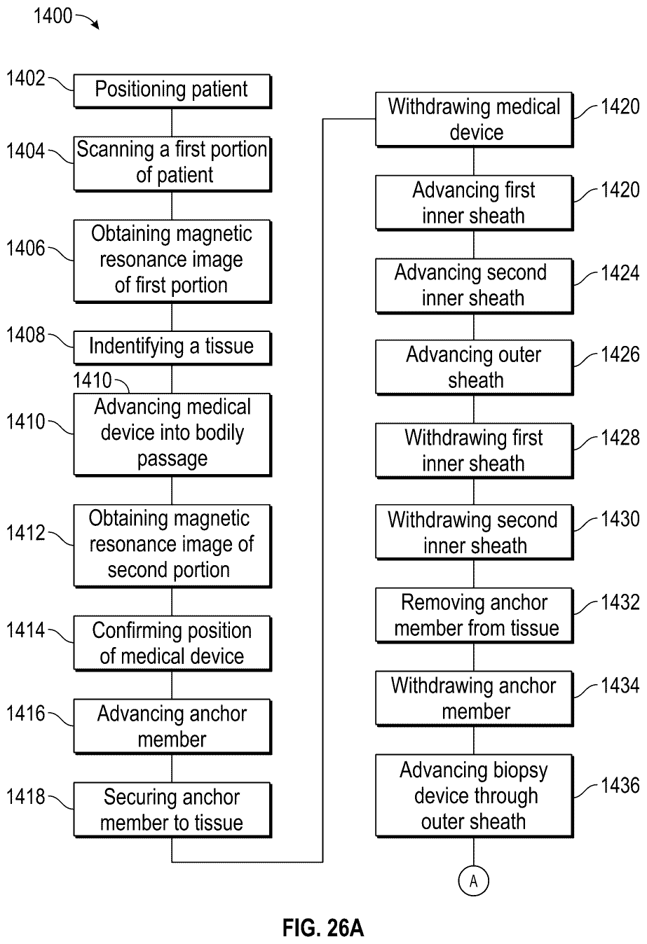

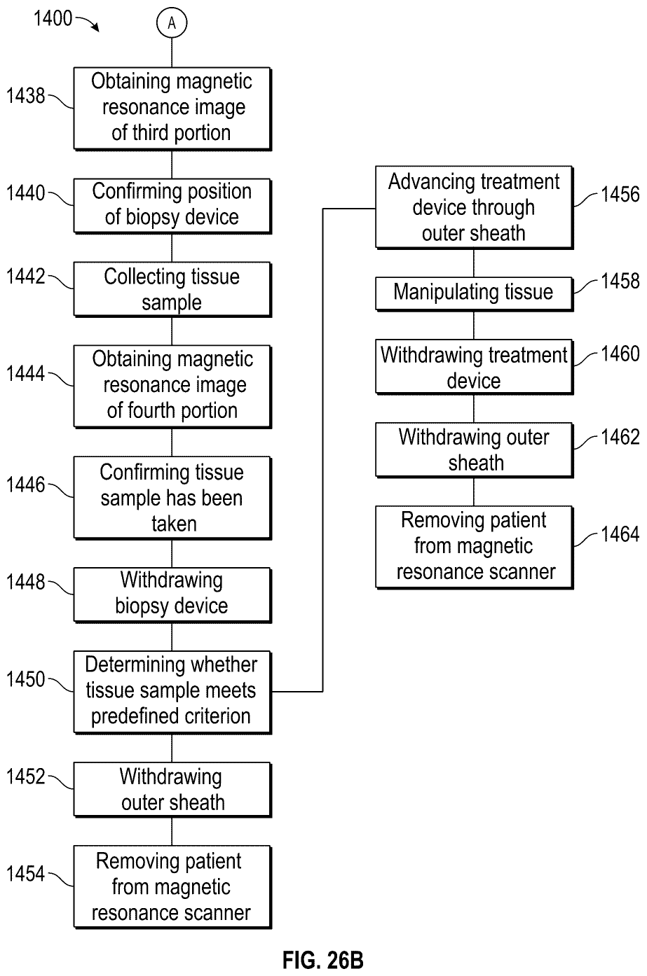

FIGS. 26A and 26B illustrate another schematic illustration of an example method 1400 of performing treatment under MRI.

An initial step 1402 comprises positioning a patient within a magnetic resonance scanner. Another step 1404 comprises scanning a first portion of the patient using the magnetic resonance scanner. Another step 1406 comprises obtaining a magnetic resonance image of the first portion of the patient. Another step 1408 comprises identifying a tissue that has predefined characteristics using the magnetic resonance image. While the patient remains positioned within the magnetic resonance scanner used to scan a portion of the patient, another step 1410 comprises advancing a medical device into a bodily passage and to, within, or adjacent to, the tissue while scanning a second portion of the patient that includes the medical device using the magnetic resonance scanner. Another step 1412 comprises obtaining a magnetic resonance image of the second portion of the patient that includes the medical device. Another step 1414 comprises confirming the position of the medical device within the bodily passage. Another step 1416 comprises advancing an anchor member through the medical device and to the tissue while the patient remains positioned within the magnetic resonance scanner. Another step 1418 comprises securing the anchor member to the tissue to retain the position of the anchor member relative to the tissue. Another step 1420 comprises withdrawing the medical device from the bodily passage. Another step 1422 comprises advancing a first inner sheath over the anchor member and toward the tissue to dilate the bodily passage. Another step 1424 comprises advancing a second inner sheath over the first inner sheath and toward the tissue to dilate the bodily passage. Another step 1426 comprises advancing an outer sheath over the second inner sheath and toward the tissue to dilate the bodily passage. Another step 1428 comprises withdrawing the first inner sheath from the bodily passage. Another step 1430 comprises withdrawing the second inner sheath from the bodily passage. Another step 1432 comprises removing the anchor member from the tissue. Another step 1434 comprises withdrawing the anchor member from the bodily passage. Another step 1436 comprises advancing a biopsy device through the outer sheath and to the tissue while scanning a third portion of the patient that includes the biopsy device using the magnetic resonance scanner. Another step 1438 comprises obtaining a magnetic resonance image of the third portion of the patient that includes the biopsy device. Another step 1440 comprises confirming the position of the biopsy device. Another step 1442 comprises collecting a tissue sample from the tissue using the biopsy device while scanning a fourth portion of the patient that includes the biopsy device and the tissue using the magnetic resonance scanner. Another step 1444 comprises obtaining a magnetic resonance image of the fourth portion of the patient that includes the biopsy device. Another step 1446 comprises confirming the tissue sample has been collected. Another step 1448 comprises withdrawing the biopsy device and the tissue sample through the outer sheath. Another step 1450 comprises determining whether the tissue sample meets a predefined criterion. If the tissue sample does not meet the predefined criterion, additional steps comprise 1452 withdrawing the outer sheath from the bodily passage and 1454 removing the patient from the magnetic resonance scanner. If the tissue sample meets the predefined criterion, another step 1456 comprises advancing a treatment device through the outer sheath and to the tissue. Another step 1458 comprises manipulating the tissue using the treatment device. Another step 1460 comprises withdrawing the treatment device from the outer sheath. Another step 1462 comprises withdrawing the outer sheath from the bodily passage. Another step 1464 comprises removing the patient from the magnetic resonance scanner.

While a number of methods have been described herein, it will be appreciated that the method may be a non-invasive method that does not require an invasive intervention by a medical professional. For example, a method may be carried out within a body lumen or passageway, such as the ear canal or a nasal passage, for example in order to place a device within such a passageway. Equally, methods may be implemented on a cadaver or artificial body parts for example for training purposes. Moreover, the skilled person will appreciate that the methods described herein may not be used on the human or animal body at all, but may be used in order to view other types of devices using MRI imaging techniques, for example in an industrial setting.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A method of performing treatment under magnetic resonance imaging comprising:
positioning a patient within a magnetic resonance scanner;
scanning a first portion of the patient using the magnetic resonance scanner;
obtaining a magnetic resonance image of the first portion of the patient;
identifying a tissue that has predefined characteristics using the magnetic resonance image;
while the patient is positioned within the magnetic resonance scanner used to scan the first portion of the patient, advancing a medical device into a bodily passage and to the tissue while scanning a second portion of the patient that includes the medical device using the magnetic resonance scanner;
obtaining a magnetic resonance image of the second portion of the patient that includes the medical device;
confirming the position of the medical device within the bodily passage;
advancing a biopsy device through the medical device and to the tissue while scanning a third portion of the patient that includes the biopsy device using the magnetic resonance scanner;
obtaining a magnetic resonance image of the third portion of the patient that includes the biopsy device;
confirming the position of the biopsy device within the bodily passage;
collecting a tissue sample from the tissue using the biopsy device while scanning a fourth portion of the patient that includes the biopsy device and the tissue using the magnetic resonance scanner;
obtaining a magnetic resonance image of the fourth portion of the patient that includes the biopsy device;
confirming the tissue sample has been collected;
withdrawing the biopsy device and the tissue sample through the medical device; and
determining whether the tissue sample meets a predefined criterion.
